(19) United States
(12) Patent Application Publication
(10) Pub. No.: US 2010/0261889 A1
(43) Pub. Date: Oct. 14, 2010

(54) PROTEIN BASED FOAMING AGENTS AND METHODS OF MAKING THEREOF

(75) Inventor: David Bressler, St.Albert (CA)

Correspondence Address: GARDNER GROFF GREENWALD & VILLANUEVA. PC, 2018 POWERS FERRY ROAD, SUITE 800, ATLANTA, GA, 30339, US

(73) Assignee: The Governors of the University of Alberta, Edmonton (CA)

(21) Appl. No.: 12/738,855

(22) PCT Filed: Sep. 30, 2008
(86) PCT No.: PCT/IB08/03811

§ 371 Date: Apr. 20, 2010

Related U.S. Application Data
(60) Provisional application No. 60/982,803, filed on Oct. 26, 2007.

(51) Int. Cl. C07K 1/00; C07K 2/00

(52) U.S. Cl. 530/407

(57) ABSTRACT

Described herein are protein based foams and methods for making the same. The foams are derived from proteins that have been hydrolysed and chemically modified such that a foam produced from the protein has improved physical properties (e.g., reduced surface tension, increased foamability and foam stability). The protein is recovered from the animal rendering process as a component of an animal by-product (e.g., bloodmeal, bonemeal or meat).

### CROSS REFERENCE TO RELATED APPLICATION

This application claims priority upon U.S. provisional application Ser. No. 60/982,803, filed Oct. 26, 2007. This application is hereby incorporated by reference in its entirety for all of its teachings.

### SUMMARY

Described herein are protein based foams and methods for making the same. The foams are derived from proteins that have been hydrolysed and modified such that a foam produced from the protein has improved physical properties (e.g., reduced surface tension, increased foamability and foam stability). The protein is recovered from the animal rendering process as a component of an animal by-product (e.g., bloodmeal, bonemeal or meat). Thus, the process described herein does not require any non-renewable feedstocks, is expected to be highly biodegradable, and utilizes an agricultural byproduct stream as a feedstock. This is especially important as the feedstocks have lost historical value with the recent concern over Bovine Spongiform Encephalopathy (BSE) and the disposal of specified risk material.

The advantages of the materials, methods, and articles described herein will be set forth-in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF FIGURES

The accompanying Figures, which are incorporated in and constitute a part of this specification, illustrate several aspects described below.

FIG. 1 shows the effect of hydrolysis time on the surface tension of 0.5% solutions of hydrolysates from bloodmeal A in different buffers (average±standard deviation, n=3).

FIG. 2 shows the effect of hydrolysis time on the surface tension of 0.5% solutions of protein precipitates from bloodmeal A in different buffers (average±standard deviation for 3-6 replicates).

FIG. 3 shows the foamability and foam stability measurement of 0.5% solutions of hydrolysates from bloodmeal A in pH 8.3 buffer after different hydrolysis times.

FIG. 4 shows the foamability and foam stability measurement of 0.5% solutions of protein precipitates from bloodmeal A in pH 8.3 buffer after different hydrolysis times.

FIG. 5 shows the foamability and foam stability of 0.5% solutions of hydrolysates (HS) and protein precipitates (PP) from bloodmeal A, after 1 hr hydrolysis, in different pH buffers.

FIG. 6 shows the effect of hydrolysis time on the surface tension of 0.5% solutions of hydrolysates from bloodmeal B in different buffers (average±standard deviation, n=3).

FIG. 7 shows the effect of hydrolysis time on the surface tension of 0.5% solutions of protein precipitates from bloodmeal B in different buffers (average±standard deviation, n=3).

FIG. 8 shows the foamability and foam stability measurement of 0.5% solutions of hydrolysates from bloodmeal B in pH 8.3 buffer after different hydrolysis times (average±standard deviation, n=3).

FIG. 9 shows the foamability and foam stability measurement of 0.5% solutions of protein precipitates from bloodmeal B in pH 8.3 buffer after different hydrolysis times (average±standard deviation, n=3).

FIG. 10 shows the foamability and foam stability of 0.5% solutions of hydrolysates (HS) and protein precipitates (PP) from bloodmeal B, after 2 hr hydrolysis, in different pH buffers (average±standard deviation, n=3).

FIG. 11 shows the foamability and foam stability of 0.5% solutions of hydrolysates (HS) and protein precipitates (PP) from bloodmeal B, after 24 hr hydrolysis, in different pH buffers (average±standard deviation, n=3).

FIG. 12 shows the effect of hydrolysis time on the surface tension of 0.5% solutions of hydrolysates of undried blood in pH 9.2 buffer (for 2% sodium hydroxide solution treated samples, average±standard deviation, n=3).

FIG. 13 shows the foamability and foam stability measurement of 0.5% solutions of hydrolysates from undried blood in pH 9.2 buffer after different hydrolysis times with 2% sodium hydroxide solution (average±standard deviation, n=3).

FIG. 14 shows the foamability and foam stability measurement of 0.5% solutions of hydrolysates from undried blood in pH 9.2 buffer after different hydrolysis times with 4% sodium hydroxide solution.

FIG. 15 shows the degree of hydrolysis of bloodmeal A protein evaluated by the OPA method.

FIG. 16 shows the degree of hydrolysis of bloodmeal B protein evaluated by the OPA method (average±standard deviation, n=3).

FIG. 17 shows the degree of hydrolysis of undried bovine blood protein evaluated by the OPA method (average±standard deviation, n=3).

FIG. 18 shows the foamability and foam stability comparison of 2-hr protein hydrolysate from bloodmeal A and Bio-soft N25-9 solutions in pH 9.2 buffer.

FIG. 19 shows the effect of 20 μM AlCl₃ on the foamability and foam stability of 0.5% 1-hr hydrolysate solutions from bloodmeal A in pH 9.2 buffer.

FIG. 20 shows the effect of 20 μM AlCl₃ on the foamability and foam stability of 0.5% 18-hr hydrolysate solutions from bloodmeal A in pH 9.2 buffer.

FIG. 21 shows the effect of GdmCl on foamability and foam stability of 0.5% 1-hr hydrolysate solutions from bloodmeal A in pH 3.0 buffer.

FIG. 22 shows the effect of denaturation by urea and DTT on foamability and foam stability of 0.5% 8-hr hydrolysate solutions from bloodmeal B in pH 8.3 buffer.

FIG. 23 shows the effect of acylation with succinic anhydride to increase protein hydrophilicity on foamability and foam stability of 0.5% 5-hr hydrolysate solutions from bloodmeal B in pH 9.2 buffer.

FIG. 24 shows the effect of C₆, C₁₂, and C₁₈ modification to increase hydrophobicity on foamability and foam stability of 0.5% 2-hr hydrolysate solutions from bloodmeal B in pH 9.2 buffer (average±standard deviation, n=3 for unmodified hydrolysate and average, n=2 for fatty acid-modified protein).

FIG. 25 shows the effect of C₄ and C₈ modification to increase hydrophobicity on foamability and foam stability of 0.5% 2-hr hydrolysate solutions from bloodmeal B in pH 9.2 buffer.

FIG. 26 shows the SDS PAGE analysis of bovine bloodmeal B hydrolysates, 10 times dilution. (1: Protein markers; 2: bloodmeal; 3: 4-hr hydrolysate; 4: 8-hr hydrolysate; 5: 12-hr hydrolysate; 6: 16-hr hydrolysate; 7: 20-hr hydrolysate; 8: 24-hr hydrolysate).

FIG. 27 shows the SDS PAGE analysis of bovine bloodmeal B hydrolysates, 100 times dilution. (1: Protein markers; 2: bloodmeal; 3: 4-hr hydrolysate; 4: 8-hr hydrolysate; 5: 12-hr hydrolysate; 6: 16-hr hydrolysate; 7: 20-hr hydrolysate; 8: 24-hr hydrolysate).

FIG. 28 shows the HPLC analysis of 6-hr bovine bloodmeal B hydrolysate. Labels indicate retention times of standards which were run separately: Ferritin, 440,000 Da; Phosphorylase b, 97,000 Da; Aprotinin, 6,512 Da; Vitamin B12, 1,355 Da.

FIG. 29 shows the HPLC analysis of bovine bloodmeal B treated with 0.2% protease and 1% lipase. Labels indicate retention times of standards which were run separately: Ferritin, 440,000 Da; Vitamin B12, 1,355 Da.

FIG. 30 shows the two-dimensional electrophoresis of bovine bloodmeal B.

FIG. 31 shows the HPLC graph for control experiment (bovine blood meal solution without enzymatic treatment)

FIG. 32 shows the HPLC graph for hydrolysate (bovine blood meal with protease treatment).

FIG. 33 shows the Foaming ability and stability of enzymatic hydrolysate [(1) 300 ml protein hydrolysates with concentration 2.25% (weight/weight); (2) foaming 3 minute at 50 volt of the provided foaming instrument; (3) legends 1 and 2 are duplicated experiments].

FIG. 34 shows the HPLC graph for modified hydrolysate bovine blood meal with protease and lipase treatment.

FIG. 35 shows the foaming ability and stability of hydrolysed protein [(1) 300 ml protein hydrolysates with concentration 2.25% (weight/weight); (2) foaming 3 minute at 50 volt of the provided foaming instrument; (3) legends 1 and 2 are duplicated experiments].

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a protein” includes a single protein or mixtures of two or more proteins.

“Optional” or “optionally” means that the subsequently described event or circumstance can or cannot occur, and that the description includes instances where the event or circumstance occurs and instances where it does not.

Described herein are methods for producing foams. The foams are produced from modified proteins using the techniques described herein. The process for modifying the proteins generally involves two steps: (1) hydrolyzing the protein and (2) chemically or enzymatically modifying the protein. Each step is discussed in detail below.

The source of the protein starting material used in the methods described herein is generally an animal by-product such as bloodmeal, bonemeal or meat that contains proteins.

Bloodmeal as referred to herein is the dried blood left over after carcassess are processed at a rendering plant. Bloodmeal is highly biodegradable. Thus, the source of protein starting material is derived from an agricultural byproduct stream. The source of the bloodmeal can come from a number of different sources involved with animal processing including, but not limited to, porcine, bovine, and poultry.

In one aspect, the method for modifying the protein comprises:

(a) hydrolysing a protein present in an animal by-product with a base to produce a hydrolysed protein; and
(b) chemically modifying the hydrolysed protein to produce a modified protein.

The first step of the process involves hydrolyzing the proteins in the animal by-product. In one aspect, the animal by-product is bloodmeal. The bloodmeal is generally solubilized followed by hydrolysis of the proteins. In one aspect, the solubilization step and hydrolysis is conducted under basic conditions. For example, the bloodmeal can be subjected to base hydrolysis for a sufficient time and temperature in order to hydrolyse the protein. The term “hydrolyse” as used herein refers to the cleavage of amide bonds in a polypeptide to produce carboxylic acid groups and amino groups. Hydrolysis of the proteins generally results in the production of proteins and peptides with varying molecular weight as well as free amino acids. Thus, the term “hydrolysed protein” is the mixture of proteins, peptides, and/or free amino acids produced by the hydrolysis of the protein(s) present in the animal by-product.

In the case when the proteins in bloodmeal are subjected to base hydrolysis, the process generally involves mixing the bloodmeal in a base such as, for example, sodium hydroxide. The solution is mixed from 0.5 hours to 24 hours at a temperature from 25° C. to 80° C. The hydrolysis time and temperature can vary by monitoring the properties of the foam surface tension, foam stability, and foamability. These properties are discussed in detail below. After the base hydrolysis step, it may be desirable to precipitate the hydrolysed proteins. In one aspect, ammonium sulfate precipitation can be used to precipitate the hydrolysed proteins.

After the base hydrolysis, the hydrolysed proteins are chemically modified. The phrase “chemically modified” is defined herein as the addition of components or incorporating additional groups on the hydrolysed protein so that one or more properties of the resulting foam are improved when compared to the unmodified protein. For example, cations such as aluminum ions can be added to the hydrolysed proteins. In another aspect, the hydrolysed protein is chemically modified by covalently attaching a hydrophobic group to the hydrolysed protein. For example, an N-hydroxysuccinimide ester of a fatty acid can react with an amino group present on the hydrolysed protein. Procedures for producing N-hydroxysuccinimide esters of fatty acids are described in the Examples below. The fatty acids can be saturated or unsaturated. Examples of fatty acid useful herein include C₂-C₂₀ fatty acids such as butyric acid, (C₄), caproic acid (C₆), caprylic acid (C₈), lauric acid (C₁₂), and stearic acid (C₁₈). Other mechanisms and techniques known in the art for covalently attaching hydrophobic groups to proteins are useful herein.

In another aspect, the method for modifying the protein comprises:

(a) hydrolysing a protein present in animal by-product to produce a hydrolysed protein; and
(b) enzymatically modifying the hydrolysed protein to produce a modified protein.

The hydrolysis step in this aspect is not limited to any particular process. Thus, the animal by-product can be subjected to base hydrolysis as described above. In one aspect, the hydrolysis step comprises enzymatic hydrolysis. In general, enzymatic hydrolysis involves mixing the animal by-product with an enzyme for a sufficient time and temperature to achieve the desired degree of hydrolysis. Techniques for measuring the degree of hydrolysis are known in the art and described in the Examples below. The amount of enzyme used can vary depending upon the enzyme selected, the desired degree of hydrolysis and reaction time, and the source of the animal by-product. In one aspect, the ratio of enzyme to bloodmeal (w/w) is from 0.1 to 10% (w/w), 0.1 to 5%, or 1 to 2%. In one aspect, the enzyme includes a protease with a specific endopeptidase function. Several commercially available enzymes could be utilized including chemotrypsin, pepsin, trypsin, protease (from Aspergillus Saitoi), Protease (from Streptomyces Griseus), Protease (from Streptomyces Caespitous), Protease (from Saureus Strain V8), and industrial proteases such as Protex 6L, Protex 7L, Protex 30L, and Protex 89L (from Genencor International Inc.). With respect to hydrolysis conditions, the animal by-product and enzyme can be mixed from 0.5 to 48 hours at a temperature from at temperatures optimized for the specific protease utilized. The pH of the reaction can modified with buffers such as carbonates, bicarbonates, and the like.

After the hydrolysis step, the hydrolysed protein is enzymatically modified. The phrase “enzymatically modified” is defined herein as the addition of components or incorporating additional groups on the hydrolysed protein in the presence of one or more enzymes so that one or more properties of the resulting foam are improved when compared to the unmodified protein.

Enzymatic modification generally involves heating a mixture of an enzyme, hydrolysed protein produced above, and one or more triglycerides for a sufficient time and temperature to modify the hydrolysed protein. The triglycerides can be purified or used as a mixture present in an oil. For example, a vegetable oil such as canola oil or tallow oil can be mixed with the hydrolysed protein and enzyme. Not wishing to be bound by theory, it is believed the triglycerides react with the hydrolysed protein and produce a covalent bond. Prior to modification, the hydrolysed protein can be purified using the techniques described in the Examples below.

In one aspect, the enzyme used for modifying the hydrolysed protein comprises a commercially available lipase such as Lipozyme TL IM. The amount of enzyme used can vary depending upon the enzyme selected and the desired degree of modification. In one aspect, the ratio of enzyme to hydrolysed protein (w/w) is from 0.1 to 10%, 0.1 to 5%, or 1 to 2%. The hydrolysed protein, enzyme, and triglyceride can be mixed from 0.5 to 48 hours at a temperature from 25° C. to 60° C. by mixing or shaking. In certain aspects, the pH of the reaction can modified with buffers such that the pH is basic.

The modified proteins produced herein are useful in producing stable foams. Techniques for producing the foams are known in the art. In one aspect, a solution of modified protein is agitated while large amounts of air are introduced into the solution. In another aspect, a finite quantity of air is introduced into a finite quantity of modified protein in solution (i.e., sparging). Here, the liquid is forced to surround the air. In a further aspect, gas can be generated in situ within a solution of the modified protein.

The foams can be used in fire fighting applications and oil recovery operations. In the case of fire fighting applications, the modified proteins can be used to produce industrial Class A fire fighting foams, which have to meet certain requirements. For example, the foams produced by the modified proteins have a surface tension less than 32 mN/m, less than 30 mN/m, or less than 28 mN/m. The foams are also stable and exhibit good foamability for extended periods of time. For example, the foam has a foam volume of 100 to 300 mL for one hour. Not wishing to be bound by theory, it is believed that longer modified peptides contribute to increased foamability and stability, and the smaller, modified peptides and modified free amino acids reduce surface tension.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Experiment 1

### Protein Hydrolysis and Surface Tension, Foamability, and Foam Stability Measurements

Initial experiments were based on bloodmeal A. In this process, bloodmeal was dissolved and base hydrolysis using a 2% sodium hydroxide solution was performed. The bloodmeal was stirred or shaken at 180 rpm for a fixed time (1 hr to 24 hr) at 55° C., and then the pH of the solution was neutralized with hydrochloric acid to stop the hydrolysis. After hydrolysis, two experimental approaches were taken. First, the surface tension and foaming properties were measured for the original hydrolysis solutions diluted in different pH buffers. Second, 60% ammonium sulfate was used to obtain dry protein precipitate, and then the protein precipitate was dissolved in different pH buffers for surface tension and foaming properties measurements.

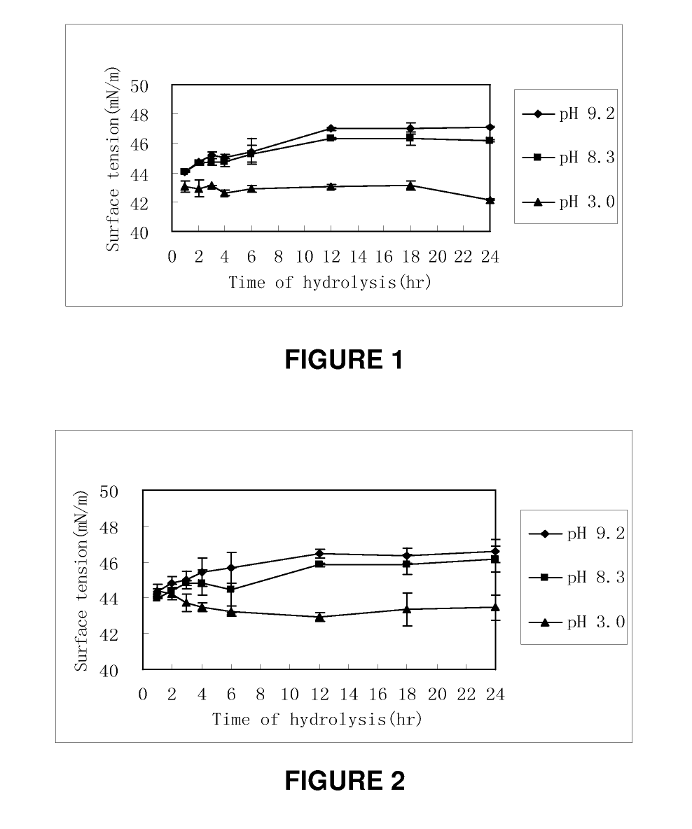

The effect of hydrolysis time and pH on the surface tension and foaming properties of the bovine proteins was investigated. The protein precipitate could not be dissolved in buffers between pH 3.0 and pH 8.3. Therefore, a pH of 3.0 was chosen as the acidic pH point, pH 8.3 as the neutral point, and pH 9.2 as the alkaline pH point. From the surface tension measurements, it was found that the surface tension went up with more hydrolysis in alkaline buffers; however, it went slightly down with more hydrolysis in acidic buffers. The data is shown in FIG. 1 for the hydrolysis solutions and FIG. 2 for the protein precipitates.

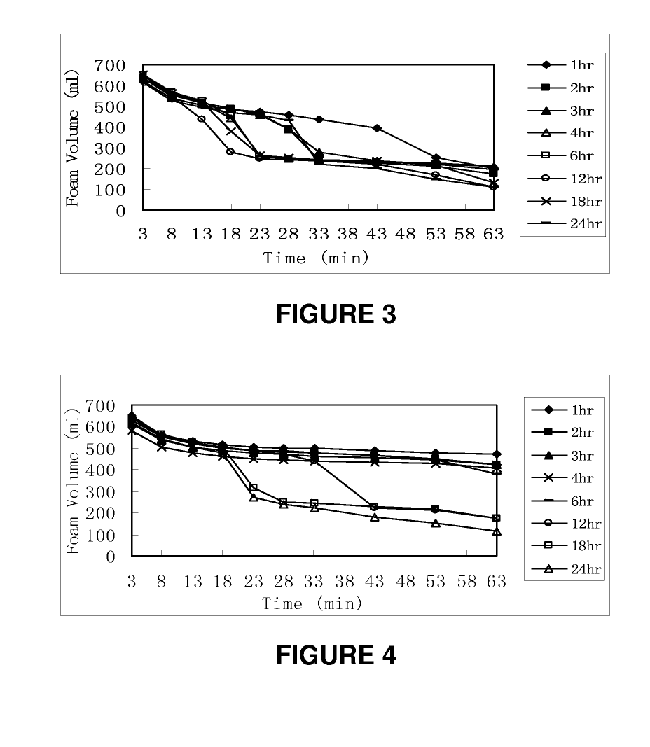

Foaming properties were measured using a foam generator provided by the Guardian industry partner. The original steel aspirator did not work consistently and was replaced by Guardian with a plastic aspirator. Although the different aspirators gave different foam volumes, it was found that the trends of all the foaming properties were the same: the foamability went up and the foam stability went down with increasing hydrolysis time. The data obtained with the steel aspirator are shown in FIGS. 3 and 4 for solutions at pH 8.3. The foams formed by either the protein precipitates or the hydrolysis solutions had very good foam stability in the higher pH buffers (pH 8.3 and pH 9.2), but they were unstable in the low pH buffer (pH 3.0) (data for pH 9.2 and pH 3.0 not shown).

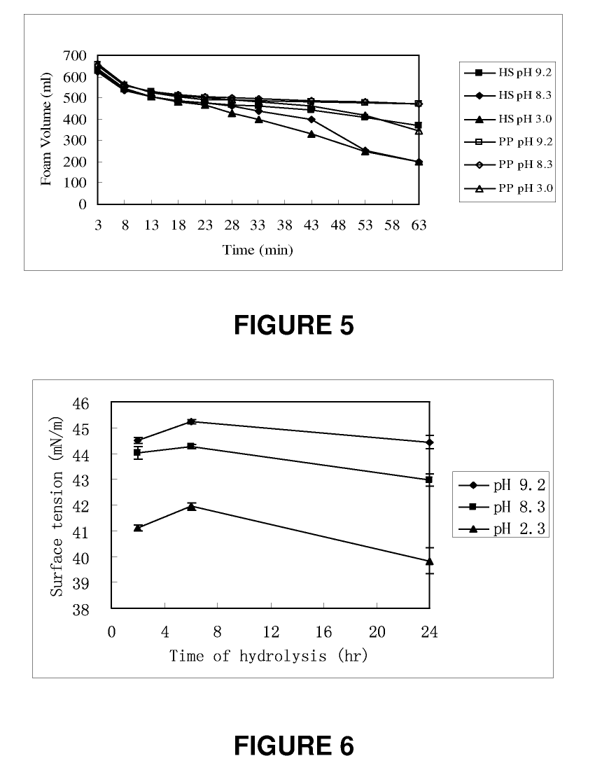

For bloodmeal A, the protein precipitate had better foamability and foam stability than the original hydrolysis solutions with the same time of hydrolysis. The data for 1-hr hydrolysis samples is shown in FIG. 5. Possible explanations are that most impurities were removed after the ammonium sulfate precipitation process. Also, not all the ammonium sulfate was removed by the filtration and the salts left in the protein precipitates might have improved the foaming properties.

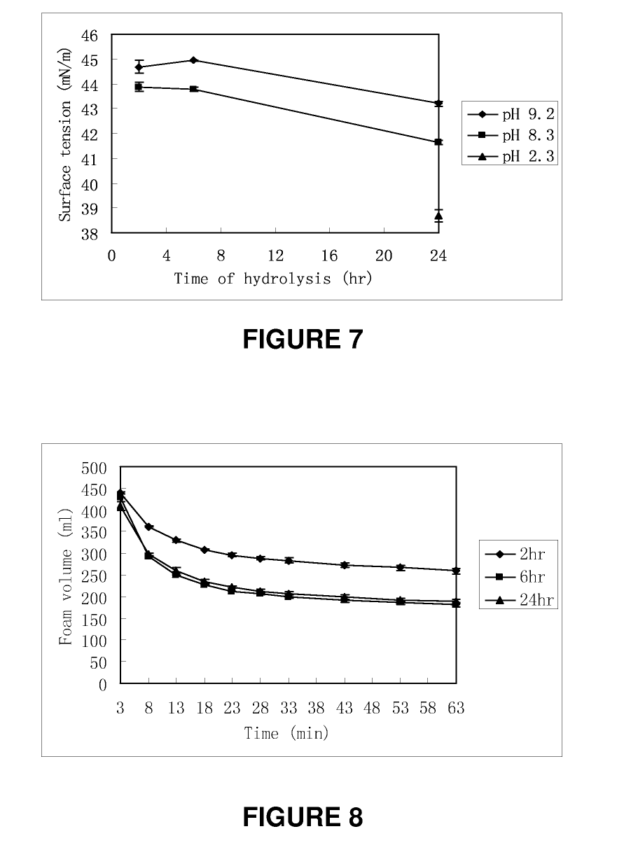

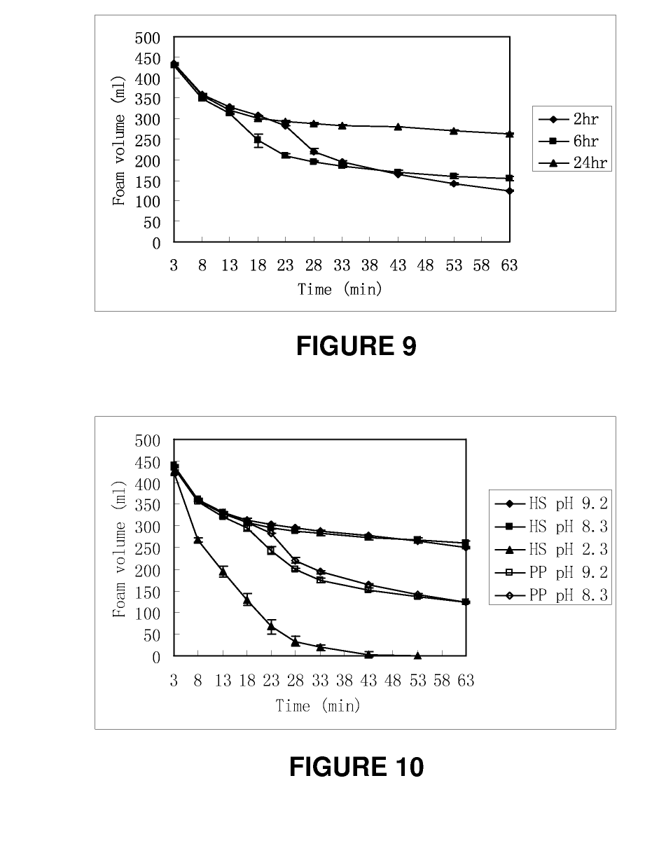

Additional experiments were carried out with bloodmeal B. The whole experimental approach was same as for bloodmeal A. The only differences were that 4% sodium hydroxide solution was used instead of 2% sodium hydroxide solution and that the temperature was increased to 60° C. during the base hydrolysis process because bloodmeal B was much harder to solubilize compared to bloodmeal A. This was anticipated due to different drying procedures and product water contents at the two source company operations. Also, the protein precipitate was no longer dissolved in pH 3.0 buffer. Only the protein precipitate after 24 hr hydrolysis could be resuspended in pH 2.3 buffer. From the surface tension measurements, it was found that the surface tension went up at the early time points and then went down with more hydrolysis. Lower pH buffers resulted in lower surface tension. The data is shown in FIG. 6 for the hydrolysis solutions and FIG. 7 for the protein precipitates.

Foaming properties were tested using the foam generator with a plastic aspirator which works consistently. The foamability and the foam stability of hydrolysis solutions went down with more hydrolysis; however, the foam stability of protein precipitates went up with increasing hydrolysis time (data for pH 9.2 and pH 2.3 not shown).

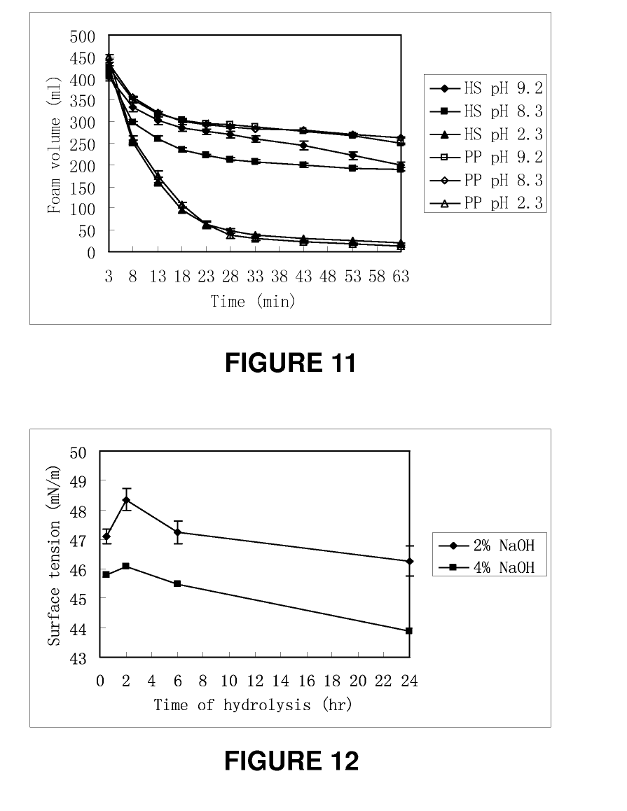

The foams formed by either the protein precipitate or the hydrolysis solutions had good foam stability in the higher pH buffers (pH 8.3 and pH 9.2), but they were unstable in the low pH buffer (pH 2.3). The foam stability of 2 hr hydrolysis solution was better than that of 2 hr protein precipitate; however, the foam stability of 24 hr hydrolysis solution was worse than that of 24 hr protein precipitate. The data for 2-hr and 24-hr hydrolysis samples is shown in FIG. 10 and FIG. 11.

Undried bovine blood was treated with both 2% sodium hydroxide solution and 4% sodium hydroxide solution at 60° C. Surface tension, foamability, and foam stability of protein hydrolysis solution in pH 9.2 buffer were measured using the same procedure. From the surface tension measurements, it was found the same trend as for bloodmeal B: the surface tension went up at the early time points and then went down with more hydrolysis. The surface tension of 4% sodium hydroxide solution treated samples was lower than that of 2% sodium hydroxide solution treated samples. The data is shown in FIG. 12.

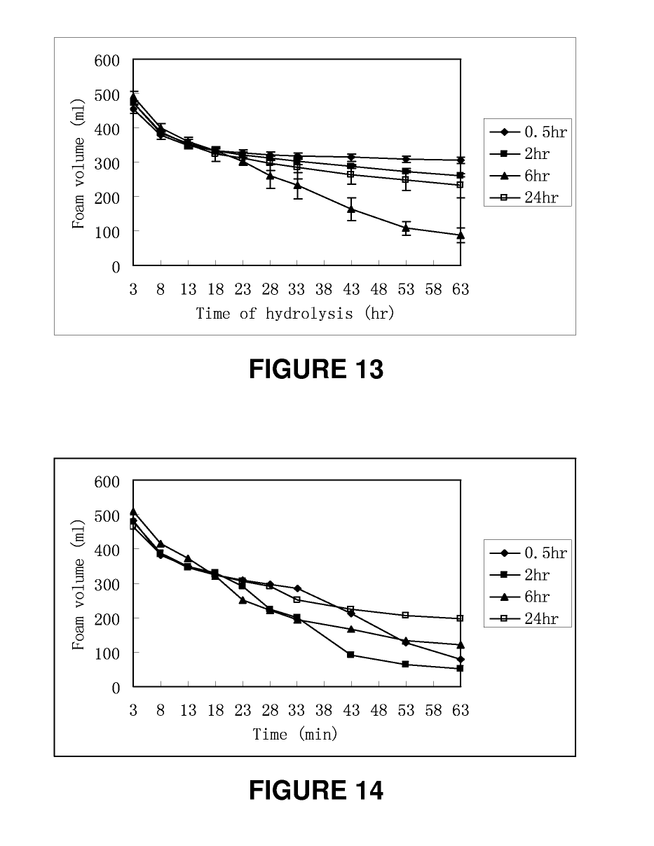

From the foaming properties measurements, it was found that the foamability went up and foam stability went down during the first several hours, and then the foam stability was increased with more hydrolysis. This cut point appeared at 6 hr for 2% sodium hydroxide solution treated samples and at 2 hr for 4% sodium hydroxide solution treated samples. The data is shown in FIG. 13 for the 2% sodium hydroxide solution treated samples and FIG. 14 for the 4% sodium hydroxide solution treated samples.

### Experiment 2

### Determination of Degree of Protein Hydrolysis by OPA Method

α-Amino groups released by hydrolysis react with o-phthaldialdehyde (OPA) and β-mercaptoethanol to form an adduct that absorbs strongly at 340 nm. During the hydrolysis reaction, 200 μl of the hydrolysate was removed from the reaction mixture at the specified time and diluted up to 2 ml with 0.1 M sodium borate buffer (pH 9.2) for the OPA assay. Diluted hydrolysate (100 μl) was added to 2 ml of OPA reagent and incubated for 2 min at room temperature (approx. 20° C.). The absorbance was measured at 340 nm with an Ultrospec 4300 spectrophotometer. The number of free amino groups was calculated by using a calibration curve of L-leucine at a similar concentration range. The increase of free amino groups was attributed to hydrolysis. The degree of hydrolysis (DH) was then calculated by the following equation:

DH (%)=[(α−nᵢ)/n_{T}]×100

where n_{T} is the total number of amino groups in native bovine proteins (estimated assuming an average molecular weight of 64,500 Da [molecular weight of Hemoglobin from bovine blood, product # H2500, http://www.sigmaaldrich.com, accessed May 15, 2007]); nᵢ is the number of free amino groups in the native protein before hydrolysis (for bloodmeal, the measurement of this number could not be conducted because bloodmeal could not be dissolved in normal buffer and water, so we assumed that nᵢ of bloodmeal was 0); and α is the number of free amino groups in the protein hydrolysate (measured).

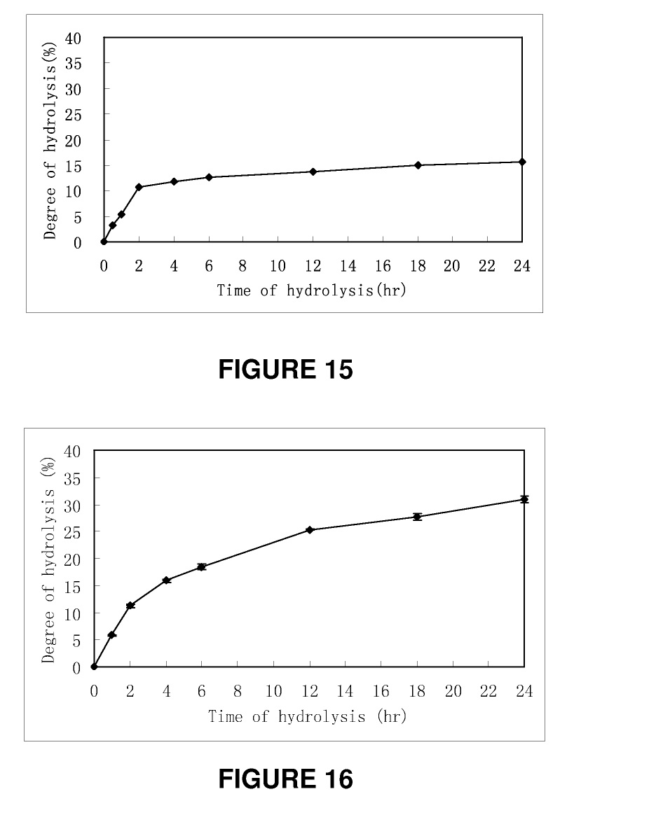

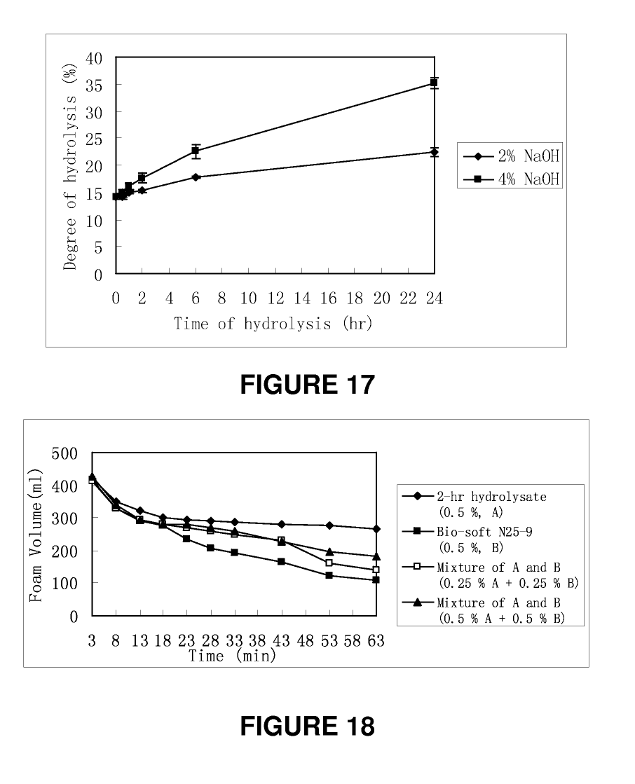

The results of the OPA assay are shown in FIG. 15 for bloodmeal A treated with 2% sodium hydroxide solution, FIG. 16 for bloodmeal B treated with 4% sodium hydroxide solution, and FIG. 17 for undried bovine blood treated with both 2% sodium hydroxide solution and 4% sodium hydroxide solution. These results indicate that the OPA assay can measure the degree of protein hydrolysis. The rate of bloodmeal protein hydrolysis was more rapid at the early time points (up to 6 hrs) than at the later times. Comparing the results of dried bloodmeal and undried bovine blood, it was found that solubilization is a rate-limiting factor for protein hydrolysis. The degree of hydrolysis after 24 hrs was not very high for bloodmeal A, likely because of the limited concentration of sodium hydroxide solution (2%) and the limited reaction temperature (55° C.). Also, the degree of hydrolysis was calculated with the assumption that the average molecular weight of bloodmeal protein was 64,500 Da. Because bloodmeal contains many different proteins, this may affect the accuracy of the results.

### Experiment 3

### Comparison of a Commercial Foaming Agent and Bovine Protein Hydrolysates

The plastic aspirator of the foam generator worked very consistently; therefore, the following experiments were carried out with the plastic one, and the steel aspirator was no longer used. A commercial foaming agent, Bio-soft N25-9, was tested as a control to compare with the bovine bloodmeal A hydrolysates. The foamability and foam stability of bovine protein hydrolysates were much better than those of the Bio-soft N25-9 (FIG. 18), while Bio-soft N25-9 gave a lower surface tension (Table 1).

TABLE 1
Surface tension comparison of 2-hr protein hydrolysate from
bloodmeal A and Bio-soft N25-9 solutions in pH 9.2 buffer

| Foaming agent | Surface tension (mN/m) |
|---|---|
| 2-hr hydrolysate (0.5%, A) | 44.84 ± 0.3 * |
| Bio-soft N25-9 (0.5%, B) | 29.75 ± 0.2 * |
| Mixture of A and B | 30.22 |
| (0.25% A + 0.25% B) | |
| Mixture of A and B | 30.21 |
| (0.5% A + 0.5% B) | |

* average ± standard deviation, n = 3

### Experiment 4

### Enhancement of the Stability of Protein-Based Foams Using Trivalent Cations

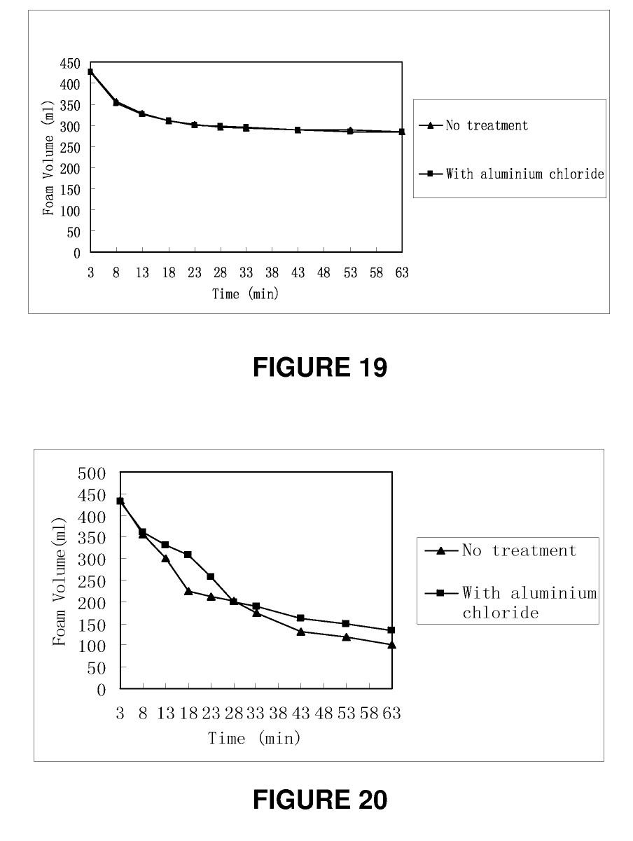

Aluminum chloride (20 μM) was added to the protein hydrolysate solutions from bloodmeal A and the surface tension and foaming properties of the samples were measured. The surface tension did not change much (Table 2). Aluminum ions did not make any difference on 1-hr protein hydrolysate (FIG. 19), but it did improve the foam stability of 18-hr protein hydrolysate (FIG. 20). Presumably as the protein molecules become smaller and smaller with increased hydrolysis, the ability of cations to cross-link the molecules is improved.

TABLE 2
Effect of 20 μM AlCl₃ on the surface tension of 0.5%
1-hr hydrolysate solutions from bloodmeal A in pH 9.2 buffer

| | |
|---|---|
| Hydrolysis | Surface tension (mN/m) |

| time (hrs) | No treatment | With AlCl₃ |
|---|---|---|
| 1 | 44.48 | 44.41 |
| 18 | 46.65 | 46.34 |

### Experiment 5

### Cleavage of Disulfide and Noncovalent Bonds of Bovine Protein Hydrolysates to Improve their Foaming Properties

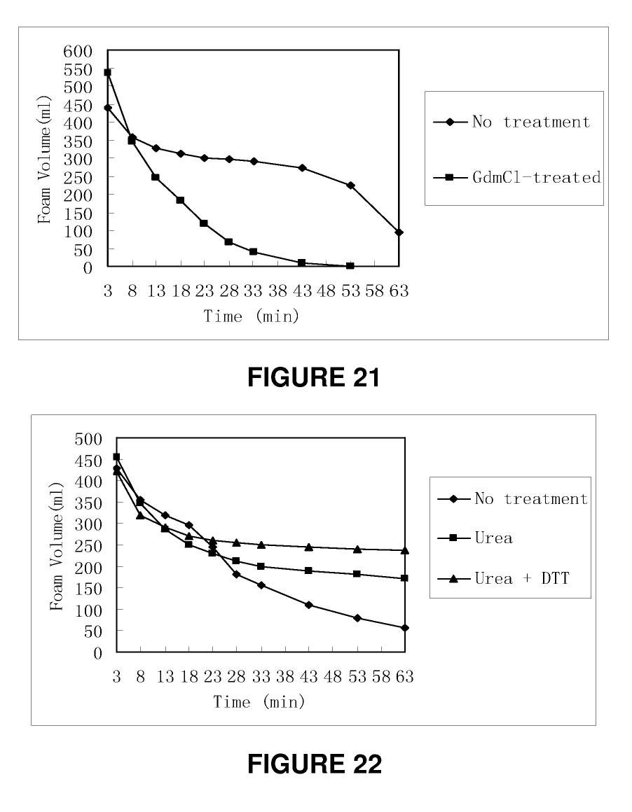

If proteins could be completely unfolded at air/water interfaces, they would have better surface-active properties. First, the disulfide bonds of the protein hydrolysate from bloodmeal A were disrupted in the presence of a denaturant (GdmCl, guanidine hydrochloride, 4 M) and thiol catalyst (3-mercaptoethanol, 0.25 mM). The protein molecules were then fixed by adding double volumes of 4% trifluoroacetic acid. Because it was an acidic environment, the 1-hr hydrolysate dissolved in pH 3.0 buffer was used as the starting material and as a control. Although this process gave a lower surface tension and increased the foam capacity, it decreased the foam stability greatly. The results are shown in FIG. 21 and Table 3.

TABLE 3
Effect of GdmCl on the surface tension of 0.5% 1-hr hydrolysate
solutions from bloodmeal A in pH 3.0 buffer

| Treatment | Surface tension (mN/m) |
|---|---|
| No treatment | 44.89 |
| GdmCl-treated | 38.61 |

Additional experiments were performed with the new bloodmeal sample B. Disulfide and noncovalent bonds restrict the unfolding of globular proteins at interfaces. In a second experiment, the protein hydrolysates from bloodmeal B were treated with urea (2 M) and dithiothreitol (DTT, 0.05 M). Urea and DTT tend to accelerate the unfolding of proteins at interfaces by disrupting most of the noncovalent bonds and reducing the disulfide bonds, respectively. Although this treatment did not change the surface tension (Table 4), it did improve the foam stability of the sample. This is shown in FIG. 22.

TABLE 4
Effect of denaturation by urea and DTT on the surface tension of
0.5% 8-hr hydrolysate solutions from bloodmeal B in pH 8.3 buffer

| Treatment | Surface tension(mN/m) |
|---|---|
| No treatment | 45.26 |
| Urea | 45.54 |
| Urea + DTT | 44.57 |

### Experiment 6

### Increasing the Hydrophilic Property of the Protein Molecules

Acylation with dicarboxylic acid anhydrides is a widely used protein modification. The anhydrides react with protein amino groups as follows (reaction 1):

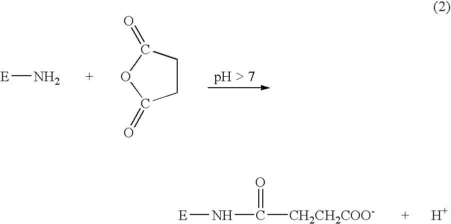

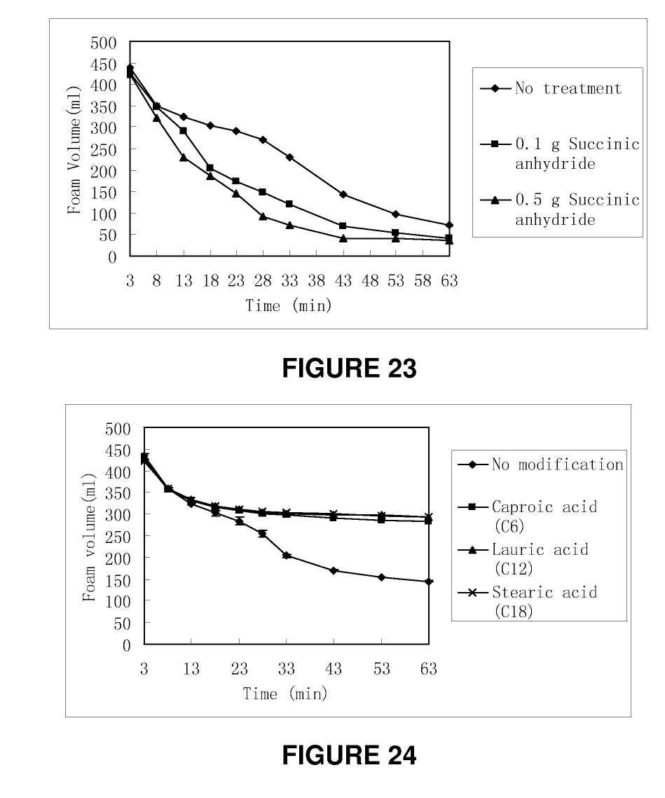

From the reaction above, a charge sign inversion is observed and the hydrophilicity of the protein molecule is increased in alkaline solution. The results in Table 5 and FIG. 23 show that the surface tension went up and the foam stability went down with succinic anhydride acylation. Therefore, increasing the hydrophilicity of the bovine protein molecules is not a suitable modification.

TABLE 5
Effect of acylation with succinic anhydride to increase protein
hydrophilicity on the surface tension of 0.5% 5-hr hydrolysate
solutions from bloodmeal B in pH 9.2 buffer

| | Surface tension |
|---|---|
| Treatment | measurement (mN/m) |
| No treatment | 45.92 |
| 0.1 g Succinic anhydride | 46.55 |
| 0.5 g Succinic anhydride | 47.73 |

### Experiment 7

### Increasing the Hydrophobic Property of the Protein Molecules

To increase hydrophobicity, modified bovine blood proteins were formed through covalent attachment of hydrophobic groups by reacting N-hydroxysuccinimide esters of various fatty acids with the amino groups. The reactions were described as follows. Fatty acids (I) reacted with N-hydroxysuccinimide (II) in the presence of dicyclohexylcarbodiimide (III) (reaction 2). The N-hydroxysuccinimide esters of fatty acids (IV) were obtained as white crystalline compounds and dicyclohexylurea (V) was removed by vacuum filtration.

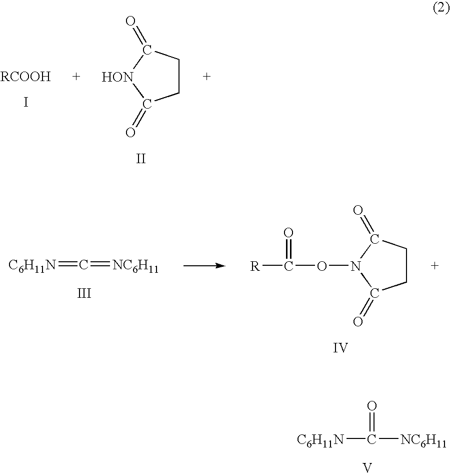

N-hydroxysuccinimide esters of fatty acids (IV) reacted with amino groups (VI) to form the corresponding N-acylamino acids (VII) (reaction 3). Through this reaction scheme, various lengths of alkyl chains were attached to the amino groups of the proteins and peptides, increasing the hydrophobicity of the protein hydrolysates.

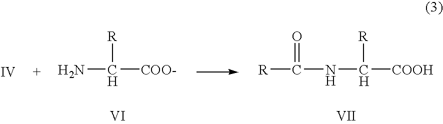

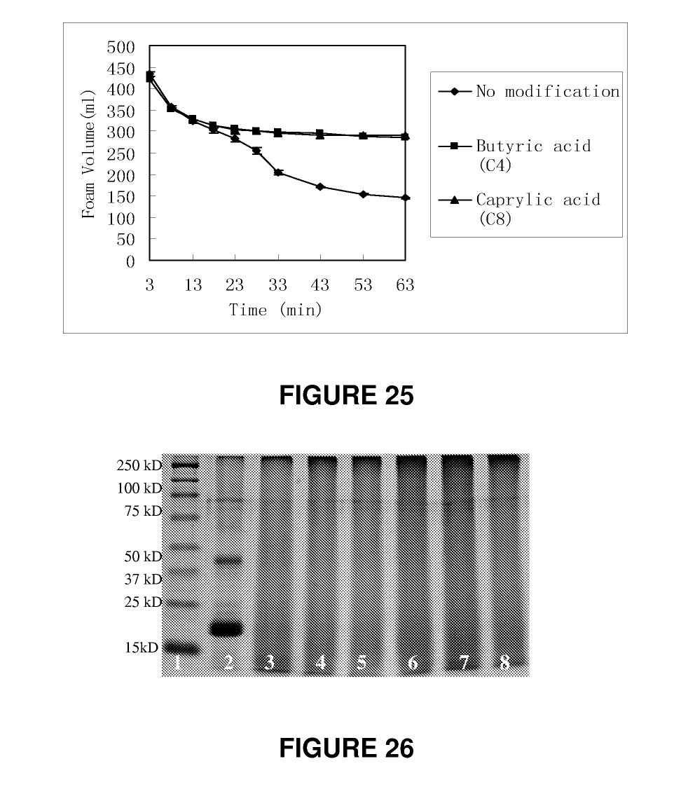

Butyric acid (C₄), caproic acid (C₆), caprylic acid (C₈), lauric acid (C₁₂), and stearic acid (C₁₈) were tested. All the fatty acids improved the foam stability of the bovine protein hydrolysates from bloodmeal B (FIGS. 24 and 25). Only C₆-modified protein decreased the surface tension to 30.77 mN/m, while the others increased the surface tension (Table 6). Tests with the C₆-modification are being repeated to ensure that this result is reproducible.

TABLE 6
Effect of increased hydrophobicity on the surface tension of 0.5%
2-hr hydrolysate solutions from bloodmeal B in pH 9.2 buffer

| Modification | Surface tension (mN/m) |
|---|---|
| No modification | 44.22 ± 0.33 * |
| Butyric acid (C₄) | 50.54 |
| Caproic acid (C₆) | 30.77 |
| Caprylic acid (C₈) | 48.29 |
| Lauric acid (C₁₂) | 46.64 * |
| Stearic acid (C₁₈) | 48.80 * |

* average ± standard deviation, n = 3 for unmodified hydrolysate and average, n = 2 for C₁₂- and C₁₈-modified protein

### Experiment 8

### Analysis of Molecular Weight Distribution in Protein Hydrolysates

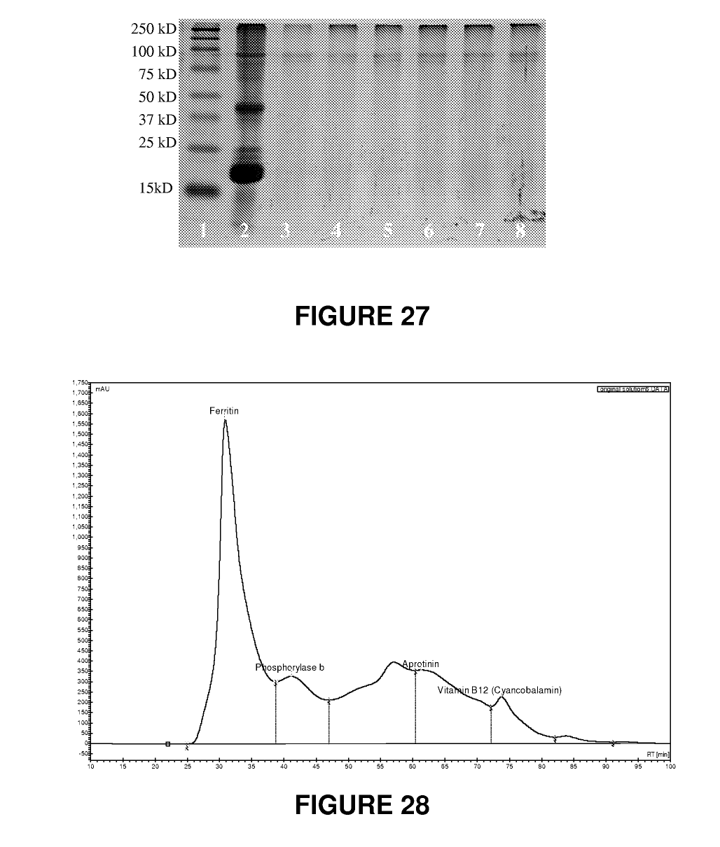

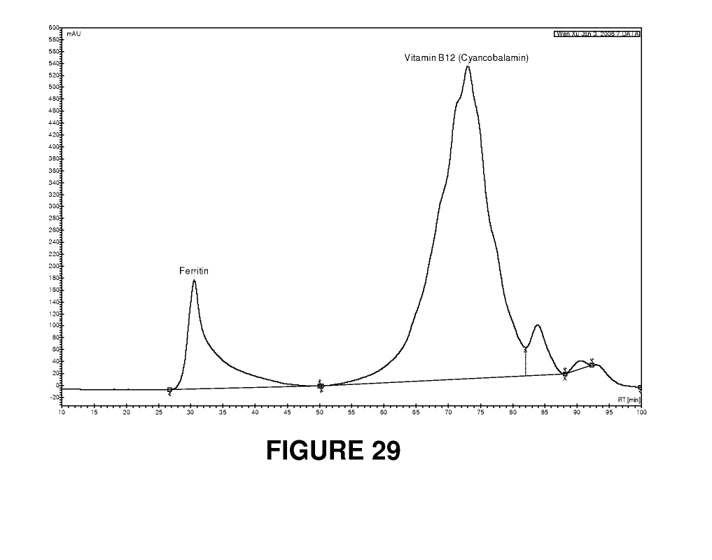

To determine the molecular weight distribution in bloodmeal B and the protein hydrolysate from bloodmeal B, high performance liquid chromatography (HPLC) connected with two high performance gel filtration columns, sodium dodecyl sulfate polyacrylamide gel electrophoresis (SDS-PAGE), and two-dimensional electrophoresis was performed. At higher concentrations, SDS-PAGE analysis of hydrolysates showed smears (FIG. 26), indicating a wide molecular weight distribution in the protein hydrolysates and confirming that cleavage of the protein molecules by base hydrolysis was random. This kind of protein cleavage is good for destroying prions, which are believed to cause Bovine Spongiform Encephalopathy (BSE). SDS-PAGE and HPLC analysis showed us there was a very large molecule which was very resistant to base hydrolysis (FIGS. 26, 27, and 28). This material had an apparent molecular weight close to 100 kDa by SDS-PAGE, and around 440 kDa by HPLC (same retention time as ferritin). Based upon experiments described below and FIG. 29, this molecule may be lipoprotein.

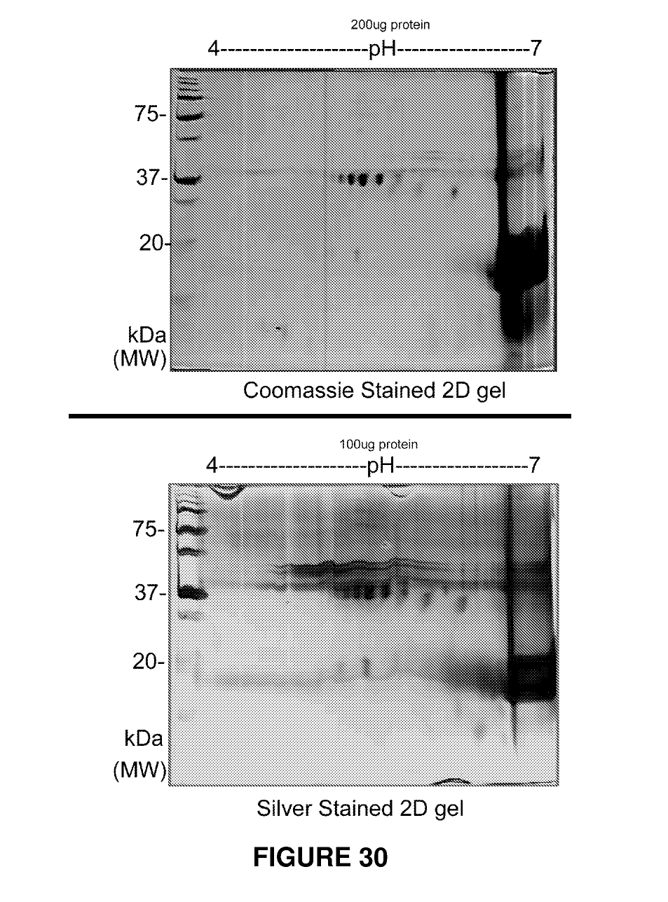

Two-dimensional electrophoresis of bovine bloodmeal did not show many spots on the gels (FIG. 30), which may be due to the protein extraction and/or the desalting process were not suitable for the proteins.

### Experiment 9

### Enzymatic Hydrolysis and Modification of Proteins

a. Experimental Method

The optimized experimental procedure for hydrolysis and modification are provided below:

Hydrolysis
1. 10 g blood meal was mixed with 300 ml buffer solution (Na₂CO₃ and NaHCO₃) at pH value 9.5.
2. Protease was added into the mixture of blood meal and buffer solution at an Enzyme: Substrate ratio (w/w) 1%.
3. Hydrolysis reaction was conducted in a shaker for 24 hours at temperature 60° C.
4. The hydrolysate was separated into supernatant and solid by centrifugation at 8000 rpm for 10 min
5. The solid was dried by vacuum oven and weighted to calculate the product recovery.
6. The supernatant was used as the hydrolysate for measurement of surface tension, and foaming ability and stability.

Modification
1. The purified hydrolysate (supernatant) was combined with canola oil at hydrolysate: canola oil ratio (volume/volume) of 10% and lipase at an enzyme: substrate ratio (weight/weight) of 1%.
2. Modification reaction was conducted in a shaker for 20 hours at temperature 60° C. and pH value 9.5.
3. The modified hydrolysate was measured for surface tension and tested for foaming ability and stability.
   b. Screening of Enzymes

During the screening of enzymes, 11 endopeptidases including chemotrysin, pepsin, trypsin, protease (from Aspergillus Saitoi), Protease (from Streptomyces Griseus), Protease (from Streptomyces Caespitous), Protease (from Saureus Strain V8), and 4 industrial proteases Protex 6L, Protex 7L, Protex 30L, and Protex 89L (from Genencor International Inc.) were tested and compared. The enzymes were applied to the bovine blood meal at an individual optimal temperature and pH value for 24 hours hydrolysis (other hydrolysis conditions were the same as description in experimental method described above. The product recovery (total solubility) was measured and used as an index for screening the enzymes and finding the suitable enzymes. Table 7 shows the comparison of product recoveries and surface tensions of protein hydrolysates from utilization of the 11 enzymes. The microbe-source proteases from Aspergillus Saitoi and Streptomyces Griseus gave higher product recovery greater than 60% and 90% respectively with high hydrolyzing ability. The industrial enzymes Protex 6L, Protex 7L and Protex 89L gave higher recovery greater than 60%, 60% and 80%, respectively. Considering the cost of enzymes, Protex 6L should be the enzyme suitable for the further research and practical production as an effective enzyme to hydrolyze blood meal. In addition, surface tension was measured to give the preliminary evaluation of hydrolysate feature. The results show that Protex 89L, Protex 6L, and Protex 30L can lead to lower surface tension from 45.097 mN/m to 49.125 mN/m among the 4 industrial enzymes. Protex 6L was chosen as the enzyme for next experiments because of its best performance in hydrolyzing blood meal and reducing surface tension.

TABLE 7
Comparison of product recoveries and mean
surface tensions of hydrolysates from hydrolysis
of bovine blood meal with proteases

| | Recovery | Mean surface |
|---|---|---|
| Enzyme | (%) | tension (mN/m) |
| Chymotrypsin | 42.4 | 49.157 |
| Pepsin | 26.0 | 46.893 |
| Protease (Aspergillus Saitoi) | 62.0 | 51.959 |
| Protease (Streptomyces Griseus) | 92.9 | 47.817 |
| Protease (Streptomyces Caespitous) | 37.5 | 50.254 |
| Protease (Saureus Strain V8) | 9.2 | 48.806 |
| Protex6L | 81.4 | 47.347 |
| Protex7L | 32.3 | 46.061 |
| Protex30L | 60.9 | 49.125 |
| Protex89L | 60.8 | 45.097 |
| Trypsin | 51.9 | 46.122 |

c. Hydrolysis with Protease and Modification with Lipase

Raw Material

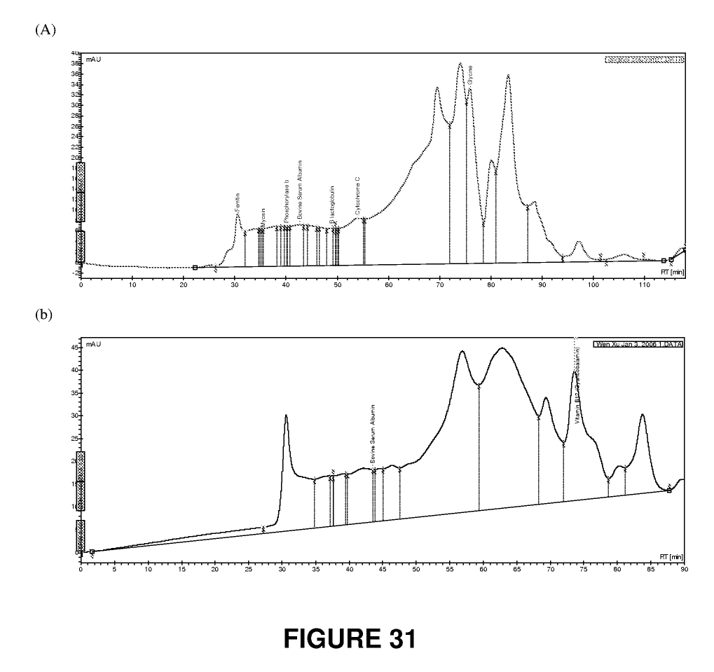

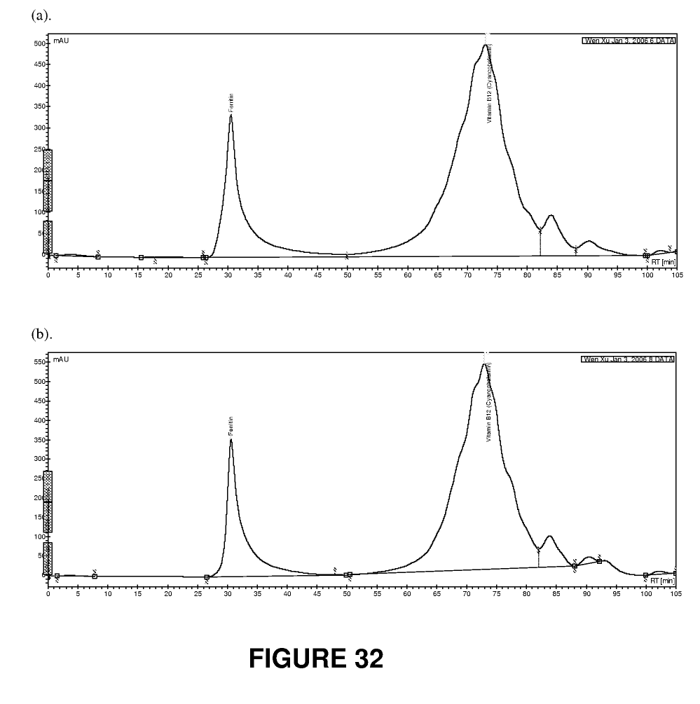

FIG. 31 ((a) and (b)) shows the high performance liquid chromatography (HPLC) graph of the soluble portion in raw material (bovine blood meal). The result shows that the general molecular weights of substances in raw material (bovine blood meal) range from larger than 1000 Dalton to less than 440 kDalton.

Hydrolysis with Protease

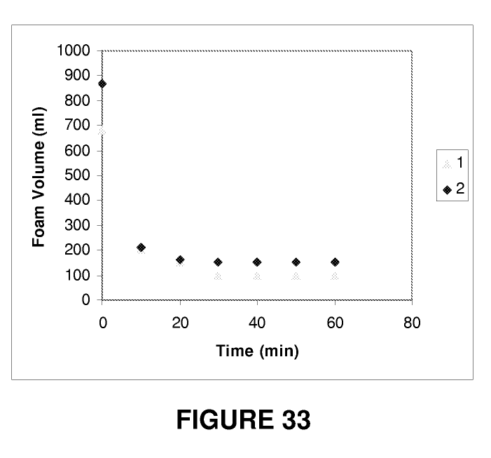

FIG. 32 ((a) and (b)) shows that the general molecular weights of amino acids and polypeptides in hydrolysate (protein hydrolysis with protease) mainly range from 7000 Dalton to 50 Dalton, which is contributed by polypeptides consisting of about 20 amino acids or single amino acid; and 480 kDalton to 200 kDalton which is contributed by polypeptides consisting of about 4000 amino acids to 1800 amino acids. The surface tension of the hydrolysate is 47.808±0.092 mN/m. FIG. 33 shows the foaming ability and stability of enzymatic hydrolysate of the bovine blood meal. The result shows that the hydrolysate has bad foaming stability.

Modification with Lipase

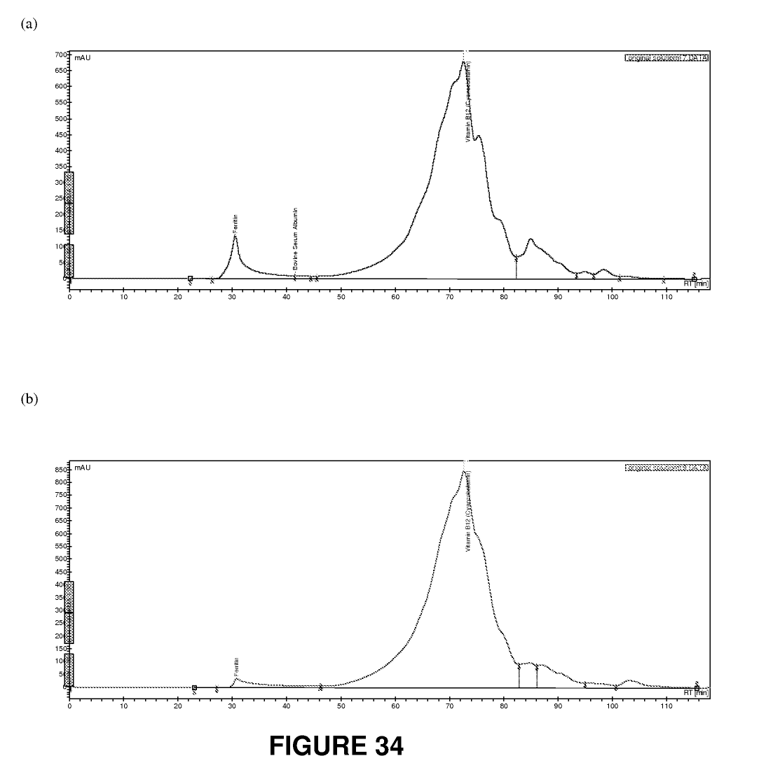

FIG. 34 shows the general molecular weight distribution of modified hydrolysate from two steps of protein hydrolysis with protease and modification with lipase. Comparing with FIG. 32, the amount of substances in the main range of molecular weights from 7000 Dalton to 50 Dalton which is contributed by polypeptides consisting of about 20 amino acids or single amino acid increases in FIG. 3, and the amount of substances in the range of molecular weights from 480 kDalton to 200 kDalton which is contributed by polypeptides consisting of about 4000 amino acids to 1800 amino acids decreases in FIG. 34. The surface tension of the modified hydrolysate is 29.040±0.018 mN/m, which is decreased from the surface tension of hydrolysate 47.808±0.092 mN/m.

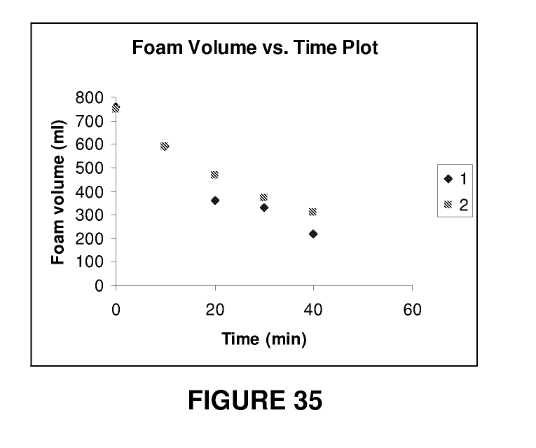

The modified hydrolysate treated with lipase shows the better result in terms of surface tension as low as 28 mN/m and a very good foaming ability as shown in FIG. 35. The foaming stability in FIG. 35 shows that there is improvement for the modified hydrolysates comparing to the foaming result of hydrolysates (FIG. 33).

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

1. A method for making a foam derived from a modified protein, wherein the modified protein is produced by the process comprising:
(a) hydrolysing a protein present in an animal by-product with a base to produce a hydrolysed protein; and
(b) chemically modifying the hydrolysed protein to produce a modified protein.

2. The method of claim 1, wherein the base hydrolysis is conducted from 0.5 to 24 hours.

3. The method of claim 1, wherein the hydrolysed protein is chemically modified by covalently attaching a hydrophobic group to the hydrolysed protein.

4. The method of claim 3, wherein the hydrophobic group is derived from a fatty acid.

5. The method of claim 4, wherein the fatty acid comprises a C₂-C₂₀ fatty acid.

6. The method of claim 4, wherein the fatty acid is caproic acid or other small small fatty acid.

7. The method of claim 1, wherein the animal by-product comprises bloodmeal.

8. A method for making a foam derived from a modified protein, wherein the modified protein is produced by the process comprising:
(a) hydrolysing a protein present in an animal by-product to produce a hydrolysed protein; and
(b) enzymatically modifying the hydrolysed protein to produce a modified protein.

9. The method of claim 8, wherein the hydrolysis step comprises enzymatic hydrolysis.

10. The method of claim 9, wherein the ratio of enzyme to animal by-product (w/w) is from 0.1 to 10%.

11. The method of claim 9, wherein the enzyme comprises a chemotrypsin, a pepsin, a trypsin, a protease, or any combination thereof.

12. The method of claim 8, wherein step (b) comprises heating a mixture of an enzyme, hydrolysed protein, and at least triglyceride for a sufficient time and temperature to modify the hydrolysed protein.

13. The method of claim 12, wherein the triglyceride is present in a vegetable oil or tallow oil.

14. The method of claim 12, wherein the triglyceride is present in canola oil.

15. The method of claim 12, wherein the enzyme comprises a lipase.

16. The method of claim 8, wherein the animal by-product comprises bloodmeal.

17. A foam produced by the method of claim 1.

18. The foam of claim 17, wherein the foam has a surface tension less than 32 mN/m.

19. The foam of claim 17, wherein the foam has a foam volume of 100 to 300 mL for one hour.

20. A foam produced by the method of claim 8.
